(19) United States
(12) Patent Application Publication
(10) Pub. No.: US 2010/0143922 A1
(43) Pub. Date: Jun. 10, 2010

(54) METHODS FOR REDUCING OVER-REPRESENTATION OF FRAGMENT ENDS

(75) Inventor: Doron Lipson, Chestnut Hill, MA (US)

Correspondence Address: LANDO & ANASTASI, LLP, ONE MAIN STREET, SUITE 1100, CAMBRIDGE, MA, 02142, US

(73) Assignee: HELICOS BIOSCIENCES CORPORATION, Cambridge, MA (US)

(21) Appl. No.: 12/616,883

(22) Filed: Nov. 12, 2009

Related U.S. Application Data
(60) Provisional application No. 61/114,136, filed on Nov. 13, 2008.

(51) Int. Cl. C12Q 1/68; C12P 19/34

(52) U.S. Cl. 435/6; 435/91.2; 435/91.5

(57) ABSTRACT

Methods for preparing fragments for nucleic acids sequence analysis that demonstrates uniform coverage across the full fragment length. The methods disclosed herein are useful for candidate gene re-sequencing wherein the detailed analysis is performed on selected, amplified regions of the genome.

### CROSS-REFERENCE TO RELATED APPLICATIONS

This application claims priority to U.S. Patent Application Ser. No. 61/114,136, filed on Nov. 13, 2008, under 35 U.S.C. §119, the contents of which are hereby incorporated by reference in their entirety.

### BACKGROUND

Traditional nucleic acid sequencing methods that rely on amplification of nucleic acids, for example, by polymerase chain reaction (PCR), typically produce nucleic acid fragments that are approximately less than 1 kb. Sequencing analysis of these fragments shows an over-representation of fragment ends relative to the internal or middle sequences within the fragment. Fragment ends are generally known sequences, and thus have little diagnostic value.

Therefore, a need exists for methods that reduce over-representation of fragment ends in a nucleic acid sample, and allow uniform sequencing across the full length of the nucleic acid fragment.

### SUMMARY

The present invention provides, at least in part, methods for preparing nucleic acid fragments for sequence analysis. In one embodiment, methods for reducing over-representation of nucleic acid fragment ends, and/or achieving uniform sequencing across the full length of the nucleic acid fragment are disclosed.

Accordingly, the invention features a method for preparing a nucleic acid sample, e.g., DNA or RNA, for sequencing. The method includes: (a) blocking the 3′ end(s) (e.g., the 3′-hydroxyl (OH) end(s)) of a nucleic acid molecule; (b) fragmenting the nucleic acid molecule to produce one or more unblocked 3′ ends (e.g., 3′-OH) of the nucleic acid fragments; (c) modifying the unblocked 3′-OH of the nucleic acid fragments; (d) anchoring the modified nucleic acid fragments to a solid support; and (e) determining at least a portion of the nucleotide sequence of the nucleic acid molecule.

In another aspect, the invention features a method for preparing a nucleic acid sample for sequencing. The method includes: (a) blocking the 3′-end(s) (e.g., the 3′-hydroxyl (OH) end(s)) of a nucleic acid molecule; (b) fragmenting the nucleic acid to produce one or more unblocked 3′ ends (e.g., 3′-OH) of the nucleic acid fragments; (c) modifying the 5′ ends and unblocked 3′ ends (e.g., 3′-OH) of the nucleic acid fragments; (d) anchoring the modified nucleic acid fragments to a solid support; and (e) determining at least a portion of the nucleotide sequence of the nucleic acid molecule.

Embodiments of the aforesaid methods may include one or more of the following features.

In certain embodiments, the nucleic acid molecule is single stranded or double stranded. The nucleic acid molecule can be produced by an amplification reaction, e.g., by polymerase chain reaction (PCR) or cloning.

In one embodiment, the blocking of the 3′-OH of the nucleic acid molecule is performed using an enzyme, e.g., a polymerase, a transferase, or a ligase, in the presence of a chain terminating nucleotide or a nucleotide analog. Exemplary nucleotide analogues include a nucleotide lacking a 3′-OH group; and a nucleotide containing an exonuclease resistant moiety (e.g., an alpha thiophosphate). In another embodiment, the blocking of the 3′-OH of the nucleic acid molecule is performed using a ligase, in the presence of a chain terminated oligonucleotide or oligonucleotide analog.

In one embodiment, the fragmenting of the nucleic acid is performed using one or more of an enzyme, a chemical or energy. In certain embodiments, the fragmenting step generates nucleic acid fragment on average less than 1000 bases, typically, between 50 to 500, 75 to 400, 100 to 300 bases in length.

In one embodiment, the modification of the unblocked 3′-OH of the nucleic acid fragments adds a defined nucleotide sequence. For example, the defined nucleotide sequence can be added using one or more of: a terminal deoxynucleotidyl transferase in the presence of a dNTP, e.g., dATP; a polyadenosine polymerase in the presence of ATP; or a ligase in the presence of a synthetic oligonucleotide. In certain embodiments, the defined nucleotide sequence is capable of anchoring and/or attaching to a solid support, e.g., via one or more of direct chemical, hybridization, and/or a binding pair (e.g., a biotin/streptavidin pair, a hapten/antibody pair or a receptor/ligand pair).

In one embodiment, the solid support used to anchor the modified nucleic acid fragments is chosen from one or more of: a bead, a microsphere, a microparticle, a microfiber, a membrane, a transparent planar surface, or a microplate.

In yet another embodiment, the sequencing method is chosen from one or more of: sequencing-by-synthesis (e.g., single molecule sequencing-by-synthesis, including real-time or otherwise); sequencing-by-ligation; or sequencing-by-hybridization. In another embodiment, the sequencing process is performed on amplified colonies originating from single molecules.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 depicts a schematic of a method used to prepare fragments for analysis via high throughput sequencing with minimal end bias.

FIG. 2 depicts an example of over-representation of 3′-ends in single molecule sequencing of a 346 bp PCR amplicon; the Y axis represents the deviation from median coverage, X axis is the position along the PCR amplicon. Gray is the (+) strand coverage; black is the (−) strand coverage. The top figure shows the standard method without 3′-OH blocks, while the bottom is an example of practicing the methods as described with 3′OH blocking before fragmentation.

FIG. 3 depicts an example of single molecule sequencing by synthesis adding cycles of labeled dNTPs.

### DETAILED DESCRIPTION

Sequencing methods which analyze nucleic acid sequences using high throughput techniques, such as sequencing-by-synthesis, sequencing-by-ligation, or sequencing-by-hybridization, may involve direct analysis of a nucleic acid sample, without any form of amplification process, for example, detection of individually optically resolved single molecules. Alternatively, these or other sequencing methods may require prior amplification of a target nucleic acid of interest in a sample. The rationale for such amplification includes, for example, the ability to isolate and analyze only a small target fraction of the total genetic material in the sample, for example, one or a few genes or gene products.

One of these methods is generally referred to as candidate gene re-sequencing (CGR). This method is important, for example, in cases where the genes of interest have been shown to be the potential causative agent of, or linked or associated with, a disease (e.g., in cancer), and thus can be used as diagnostic or progonostic markers for disease progression and ongoing monitoring of disease remission.

Target amplification (e.g., by PCR) generally produces short fragments of about 100-500 bases or up to a few kilobases in length. The nucleic acid targets are the exons of genes and may include intron areas of known function, such as transcription start sites, regulatory domains, etc. In some cases, only one or a few gene exons are amplified, while, in other cases, the entire gene is amplified.

Following amplification, sequencing methods which are based on generating short reads, e.g., <200-300 bases, and sometimes <50 bases, normally require sample preparation methods that fragment the target nucleic acid material to similar lengths, about <300-500 bases, and more desirable <200 bases. Desirable methods of fragmentation should also produce a partial or totally random pattern of fragmentation, such as by shearing by sonication and/or limited DNase treatment.

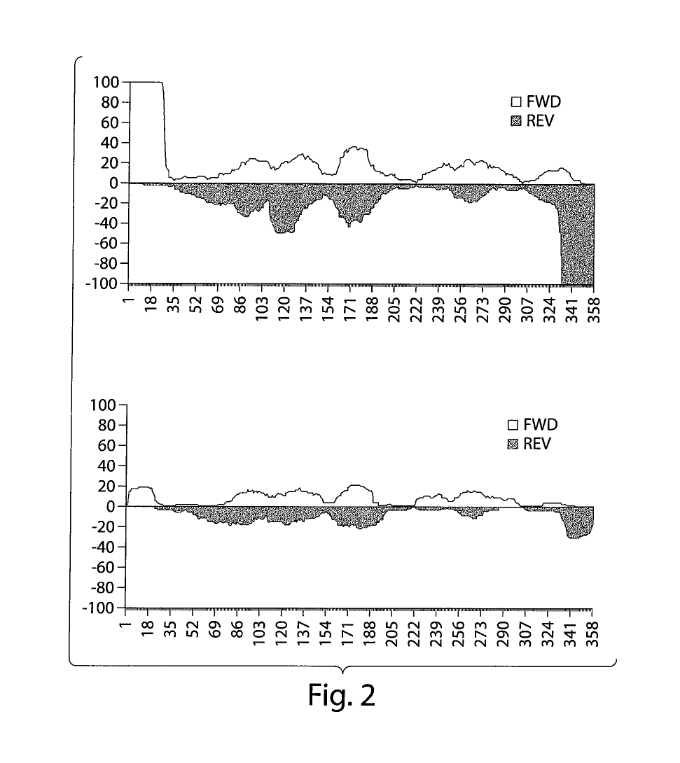

One problem associated with the amplification of a nucleic acid sample (e.g., by, PCR) to produce fragments which are <1 kb, is that upon fragmentation and subsequent sequencing analysis, the results show an over-representation of fragment ends rather than internal sequences, as depicted in FIG. 2. Additionally, the fragment ends generally correspond to a known sequence that typically has little diagnostic value. When the method of amplification used is PCR, the ends are the primers used. The mechanism underlying this observation is that when amplification products are short, e.g. 200-1000 bases, the fragmentation methods known on average only break these short pieces in a few locations or not at all, e.g. zero to 4 break points. Physical processes, e.g., sonication, which shear nucleic acids generally do not produce breaks near the ends of nucleic acid fragments. Thus, it is difficult to obtain random sequence information from the internal or middle of the nucleic acid fragments where some or all of the important diagnostic or other information may reside. This is especially important when the sequencing method used to obtain the sequence data only produces reads which are on average <50 bases in length.

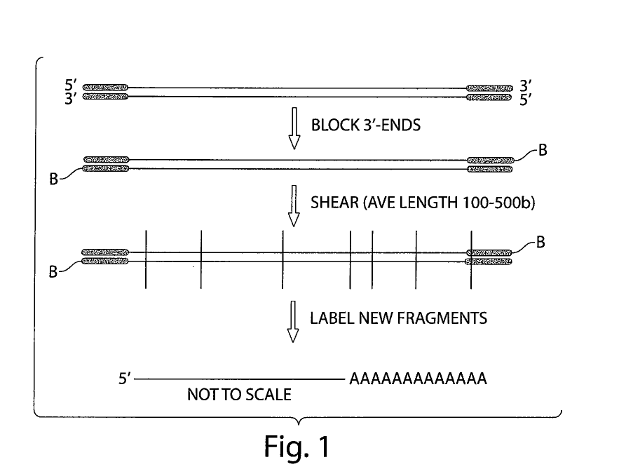

The following method, as illustrated in FIG. 1, has been found to substantially eliminate or reduce fragment-end bias in a nucleic molecule. The method relates to preparing a nucleic acid sample for sequencing, including the steps of:
a. blocking the 3′-end of a nucleic acid molecule;
b. fragmenting the nucleic acid molecule to produce one or more unblocked 3′-ends;
c. modifying the one or more unblocked 3′-ends;
d. attaching the modified nucleic acid fragments to a solid support; and
e. determining at least a portion of the sequence of the nucleic acid.

Generally, the nucleic acid molecule being analyzed is generated by PCR. Other in vitro or in vivo amplification methods are also possible, as long as the starting nucleic acid is generally <1 kilobase, and, preferably, between 50-500 bases.

Following amplification, addition of a blocker to the 3′-end of the strand (if single stranded), or the two 3′-ends (if double stranded) is done using either an enzyme or chemical modification. The purpose is to modify the 3′-OH of the nucleic acid molecule, so that it is no longer reactive to methods that generally utilize polymerase, transferase, or ligase. The blocker can take many forms, including, but not limited to, addition of nucleotide lacking a 3′-OH. Examples of such nucleotides include: 2′3′-dideoxynucleotides, 3′-deoxynucleotides, 3′-aminodeoxynucleotides, 3′-azidodeoxynucleotides, acyclonucleotides, 3′-fluorodeoxynucleotides, etc. The 2′-position can be either —OH or —H. Nucleotides are added to the amplification product using either a polymerase, a transferase, or a ligase. The enzymes can be specific for DNA or RNA. Additionally, multiple base entries, e.g., oligonucleotides or analogs, can be added onto the amplification product as a means to add a blocker.

An example of chemical modification, is when the amplification product is RNA. The RNA may be treated with periodate to cleave the 2′,3′-vicinal diols of the ribose to form aldehydes. Optionally, the diols once converted to aldehyde may be reduced. Neither of these forms allows a further base addition by a polymerase or a ligase.

The nucleotides used to block the amplification products may also include moieties that make the blocked product resistant to further enzyme action (for example nuclease action). Art-recognized modified nucleotides can be used, for example, a thiophosphate moiety at the alpha-phosphate, e.g., PO₃—O—PO₂—O—PSO—O-5′C. Other mechanisms might involve modifying the P—O-5′-C bond to some other group such as P—N-5′-C, P—S-5′-C, or P—C-5′-C.

Following blocking of the ends, random fragmentation can be performed as is standard in the art. Such methods typically include: sonication, enzymatic or chemical treatment. Following this fragmentation, it may be required to perform end repair to produce viable 3′-ends (have a functional 3′-OH) or not. Samples following fragmentation may be left as double stranded or denatured to produce single strands before subsequent modifications are performed.

Following fragmentation, the sample can be anchored to a surface in preparation for sequencing. Additional modifications may or may not be required. However, a preferred method involves attachment of a defined sequence onto each of the fragments generated. Nucleotide sequences may be added onto either the 5′ or 3′ end of the nucleic acid fragment. One preferred position of attachment is the 3′ end. Sequences added to the 5′ end are generally added by ligation based methods. The primary purpose of such sequence is to attach a sequencing primer binding site and/or enable anchoring of the fragments via hybridization. Alternatively, the fragments may be labeled in such a way as to provide anchoring to the surface via direct or indirect mechanisms, e.g., direct may include covalent attachment, and indirect may include anchoring via a binding pair and/or a polymerase, which itself may be directly or indirectly anchored. The defined sequence may be, generally, a single, unique sequence comprised of 2 or more bases attached to all fragments or a homopolymeric sequence comprised of only a single base. Generally, the sequence will be 20-70 bases in length, preferably 30-50 bases.

A method of attaching a unique nucleotide sequence to the nucleic acid fragments is using a ligase. The ligation may be blunt-ended or via overhanging ends. Ligation may also be achieved via single stranded to single stranded, using for example CircLigase™ or RNA ligase.

In embodiments where homopolymeric sequences are added, an enzyme (such as terminal deoxynucleotidyl transfer or polyA polymerase) is used. A single nucleotide, dATP or ATP, is then used to produce the homopolymeric tail. Control of the average length of A's added is by reaction control of the molar excess of (d)NTP over fragment 3′-ends.

Additionally, in one embodiment, samples from many different sources are mixed and analyzed together. In this case, the sequences used to anchor the fragments to a surface may also be encoded, so as to be able to discriminate which sequences come from which sample.

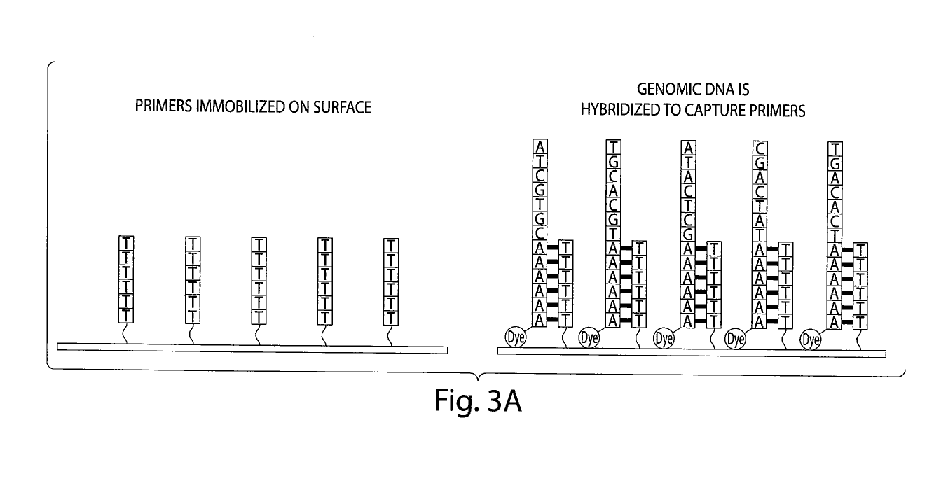

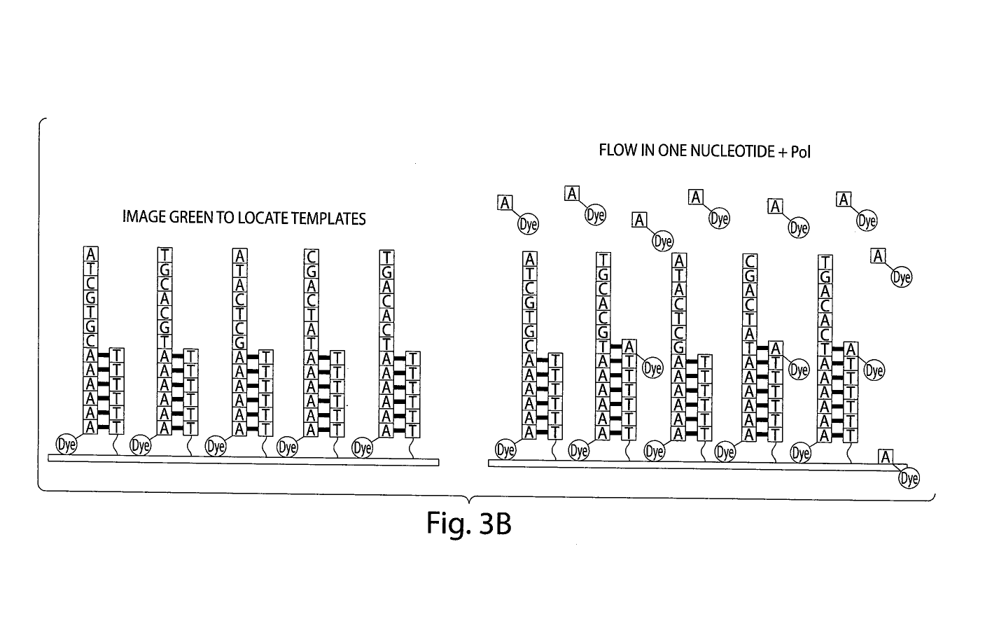

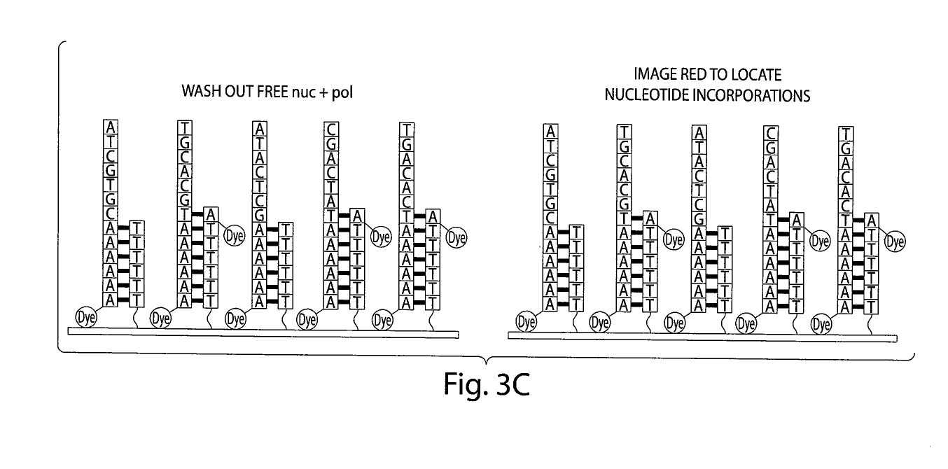

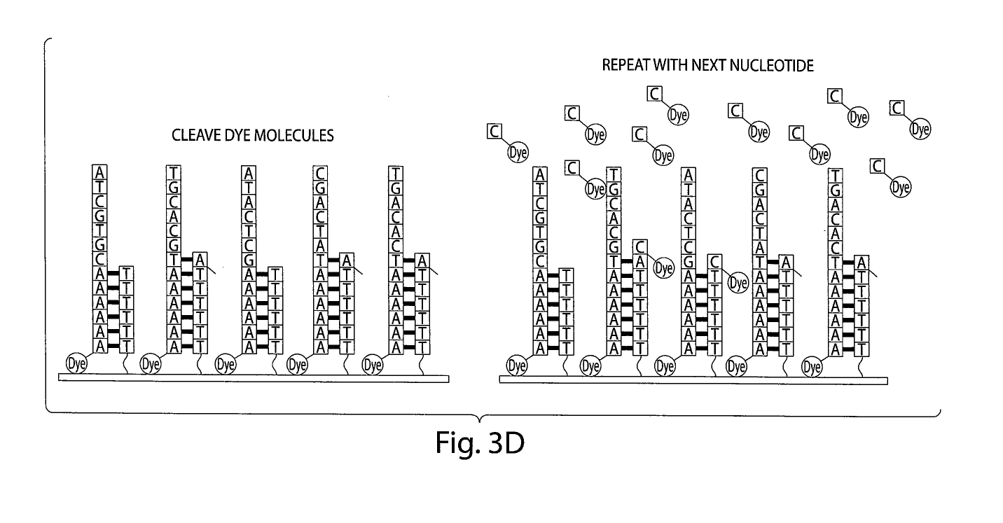

Once fragments are end labeled and anchored to a surface, four major high-throughput sequencing platforms are currently available and can be used: the Genome Sequencers from Roche/454 Life Sciences (Margulies et al. (2005) Nature, 437:376-380; U.S. Pat. Nos. 6,274,320; 6,258,568; 6,210,891), the 1G Analyzer from Illumina/Solexa (Bennett et al. (2005) Pharmacogenomics, 6:373-382), the SOliD system from Applied Biosystems (solid.appliedbiosystems.com), and the Heliscope™ system from Helicos Biosciences (see, e.g., U.S. Patent App. Pub. No. 2007/0070349, the entire disclosure of which is hereby incorporated herein by reference for all purposes, and the illustration in FIG. 3). Although these new technologies are significantly less expensive than the traditional methods, such as gel/capillary Gilbert-Sanger sequencing, the sequence reads produced by the new technologies are generally much shorter (−25-40 vs. −500-700 bases). A real-time sequencing-by-synthesis method is also under development by Pacific BioSciences.

An example of asynchronous single molecule sequencing-by-synthesis is illustrated in FIG. 3. As shown, oligonucleotides 30-50 bases in length are covalently anchored at the 5′ end to glass cover slips. These anchored strands perform two functions. First, they act as capture sites for the target template strands, if the templates are configured with capture tails complementary to the surface bound oligonucleotides. They also act as primers for the template-directed primer extension that forms the basis of the sequence reading. The capture primers are a fixed position site for sequence determination. Each cycle consists of adding the polymerase-labeled nucleotide analog mixture, rinsing, optically imaging the field containing millions of active primer template duplexes, and chemically cleaving the dye-linker to remove the dye. The labeled nucleotides are added either individually in a cycle or if the detectable moiety is spectrally resolvable more than one nucleotide can be added per cycle. The nucleotide analogs are such that they add only once per strand/cycle, e.g., a reversible terminator. The cycle (synthesis, detection, and dye removal) is repeated up to 25, 50, 100 times and, possibly, more.

The real-time single molecule sequencing-by-synthesis technologies rely on the detection of fluorescent nucleotides as they are incorporated into a nascent strand of DNA that is complementary to the template being sequenced. This type of detection depends, at least in part, upon the ability of the imaging system to differentiate which of the four spectrally resolvable fluorescent nucleotides in the polymerase-labeled nucleotide mixture incorporates as the polymerase copies the template in near real time.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

All references cited herein are incorporated herein by reference in their entirety and for all purposes to the same extent as if each individual publication or patent or patent application was specifically and individually indicated to be incorporated by reference in its entirety for all purposes.

### EQUIVALENTS

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

We claim:

1. A method for reducing over-representation of nucleic acid fragment ends, comprising:
a. blocking the 3′-OH of a nucleic acid molecule;
b. fragmenting the nucleic acid molecule to produce one or more unblocked 3′-OH;
c. modifying the one or more unblocked 3′-OH;
d. anchoring the modified nucleic acid fragments to a solid support; and
e. determining at least a portion of the sequence of the nucleic acid molecule.

2. The method of claim 1, wherein the nucleic acid molecule is DNA or RNA.

3. The method of claim 1, wherein the nucleic acid molecule is single stranded or double stranded.

4. The method of claim 1, wherein the nucleic acid molecule is produced by an amplification reaction.

5. The method of claim 4, wherein the amplification process is polymerase chain reaction (PCR) or cloning.

6. The method of claim 1, wherein the blocking is performed using an enzyme in the presence of a chain terminating nucleotide or nucleotide analog.

7. The method of claim 6, wherein the enzyme is chosen from a polymerase, a transferase, or a ligase.

8. The method of claim 6, wherein the nucleotide lacks a 3′-OH or additionally contains an exonuclease resistant moiety.

9. The method of claim 8, wherein the nucleotide contains an alpha thiophosphate.

10. The method of claim 1, wherein the blocking step is performed using a ligase in the presence of a chain terminated oligonucleotide or oligonucleotide analog.

11. The method of claim 1, wherein the fragmenting step is performed using an enzyme, a chemical or energy.

12. The method of claim 11, wherein the fragmenting step generates fragment lengths on average between 50-500 bases.

13. The method of claim 1, wherein the modification of the unblocked 3′-OH adds a defined sequence.

14. The method of claim 13, wherein the defined sequence is added using terminal deoxynucleotidyl transferase in the presence of a dNTP.

15. The method of claim 14, wherein the dNTP is dATP.

16. The method of claim 13, wherein the defined sequence is added using polyadenosine polymerase in the presence of ATP.

17. The method of claim 13, wherein the defined sequence is added using a ligase in the presence of a synthetic oligonucleotide.

18. The method of claim 13, wherein the defined sequence is attached or anchored to a solid support.

19. The method of claim 1, wherein the anchoring to a support is effected by a direct or indirect mechanism including one or more of a covalent bond, a hybridization, a polymerase, or via a binding pair, including any combinations thereof.

20. The method of claim 19, wherein the binding pair is a biotin/streptavidin pair, a hapten/antibody pair or a receptor/ligand pair.

21. The method of claim 1, wherein the solid support is a bead, a microsphere, a microparticle, a microfiber, a membrane, a transparent planar surface, or a microplate.

22. The method of claim 1, wherein the sequencing method is chosen from one or more of: sequencing-by-synthesis, single molecule sequencing-by-synthesis, sequencing-by-ligation or sequencing-by-hybridization.

23. The method of claim 1, wherein the sequencing process is performed on amplified colonies originating from single molecules.

24. A method for reducing over-representation of nucleic acid fragment ends, comprising:
a. blocking the 3′-end of a nucleic acid molecule;
b. fragmenting the nucleic acid molecule to produce one or more unblocked 3′-OH;
c. modifying both 5′ ends and one or more unblocked 3′-OH;
d. anchoring the modified nucleic acid fragments to a solid support; and
e. determining at least a portion of the sequence of the nucleic acid molecule.

25. The method of claim 24, wherein the sequencing process is performed on amplified colonies originating from single molecules.

26. The method of claim 24, wherein the solid support is a bead, a microsphere, a microparticle, a microfiber, a membrane, a transparent planar surface, or a microplate.

27. The method of claim 24, wherein the sequencing method is chosen from one or more of: sequencing-by-synthesis, single molecule sequencing-by-synthesis, sequencing-by-ligation or sequencing-by-hybridization.
